(19) United States
(12) Patent Application Publication
(10) Pub. No.: US 2008/0163876 A1
(43) Pub. Date: Jul. 10, 2008

(54) DEVICE FOR TRAINING WEARER WRIST POSITION AND FOR REMINDING WEARER OF WRIST POSITION

(76) Inventor: Eileen J. Phelan, Portland, OR (US)

Correspondence Address: PATENTTM.US, P. O. BOX 82788, PORTLAND, OR, 97282-0788, US

(21) Appl. No.: 11/621,741

(22) Filed: Jan. 10, 2007

(51) Int. Cl. A61F 5/37; A61F 5/00

(52) U.S. Cl. 128/879; 602/21

(57) ABSTRACT

A safety device does not prevent any movement of the hand, wrist, or fingers; but discourages extension, the upward movement of the wrist. A “breakaway” property is provided so that the wearer may have complete freedom at any point. The device is worn to keep the user's wrist in a safe position least likely to induce carpal tunnel syndrome, reminding the user to hold the hand in a position which may relieve carpal tunnel syndrome or reduce the likelihood of acquiring this syndrome.

### BACKGROUND OF THE INVENTION

This invention relates to wrist function and more particularly to a device to reduce the likelihood of developing or to minimize movement that would exacerbate symptoms of Carpal Tunnel Syndrome.

Carpal Tunnel Syndrome is a widely known and widely experienced repetitive stress injury. The incidence is increasing as more people are using computer keyboards for a significant period of time during the day.

A number of devices exist for the purposes of preventing and treating carpal tunnel syndrome. Most of these devices restrict the movement of the wearer; some are designed to themselves provide therapeutic treatment. All the devices are completely and firmly affixed to the wrist. Most prevent flexion, although the typical computer user holds his or her hands in a potentially harmful position of wrist extension.

The prior art consists of 3 main types of devices: devices which immobilize, therapeutic devices, and finger restraints.

Devices Which Immobilize

These devices immobilize the wrist and do not break away; my invention allows full wrist flexion. It discourages wrist extension but does not prevent it, and breaks away with slight pressure.

U.S. Pat. No. 4,941,460 (Working): this device “prevents flexion, hyperextension, and ulnar deviation of the wearer's wrist”.

U.S. Pat. No. 5,368,550 (Sisley): this includes an outer shell and an inner liner, . . . “provided for limiting an amount of flexion of the human joint when the strap secures the outer shell around the human joint.”

U.S. Pat. No. 5,417,645 (Lemmen): “The splint . . . provides increased resistance to bending of the wrist the further the wrist is bent from the normal cocked up position.”

U.S. Pat. No. 5,456,650 (Williams, Jr. et al.): This is a sports device which “specifically relates to the use of rods in a glove to offer resistance to individual fingers . . . and the use of a brace for holding the wrist in place . . . ”

U.S. Pat. No. 5,672,150 (Cox): This is “An opposing force wrist brace . . . that immobilizes the wrist . . . ”

U.S. Pat. No. 6,517,501 B1 (Slautterback): This device is designed for “ensuring a neutral hand position of 15 degrees at all times when worn . . . ”

U.S. Pat. No. 6,740,056 B2 (Slautterback): This invention is intended “to restrict motion of the wrist which is related to carpal tunnel syndrome.” It “restricts flexion of the hand between the hypothenar eminence and the center of thenar eminence.” This “keeps the wrist and hand from drooping below the neutral position.”

Therapeutic Devices

The following devices, which work according to differing methods, are in and of themselves therapeutic;

U.S. Pat. No. 5,385,537 (Davini): “ . . . The strap 17 is then exteriorly wrapped around the ulna 16 and is pulled enough to cause a proper support of the wrist 14 and to cause a slight approximation of the radius 15 and the ulna 16 toward one another.”

U.S. Pat. No. 5,468,220 (Sucher): This is an “appliance for opening the carpal canal by stretching the transverse carpal ligament and the surrounding structure . . . ”

U.S. Pat. No. 5,766,141 (Gould): This is “A wrist brace for correction of carpal tunnel syndrome . . . ” which provides “strategically located leverage and pressure points to relieve the restriction of the medial nerve . . . ”

U.S. Pat. No. 5,919,151 (Gustafson): This device is constructed “so as to provide a generally dorsally directed force to the palm and thereby maintain the flexor tendon to the desired position so as to resist median nerve compression in the carpal tunnel”.

U.S. Pat. No. 6,120,472 (Singer, Jr.): This system “reduces pressure in the carpal tunnel by rotating the radius and ulna towards each other, thus reducing tension in the flexor retinaculum and transverse carpal ligament.”

U.S. Pat. No. 6,217,536 B1 (Gustafson): This invention causes “reduction of median nerve compression in the carpal tunnel.”

U.S. Pat. No. 6,723,061 B2 (Williams): This is “a splint for providing dynamic pressure to the transverse carpal, volar carpal, and intra-carpal ligaments, in a manner tending to relieve contractures of these ligaments . . . ”

2002/0035342 A1 (Williams): This is a dynamic splint which provides “a continuous, low level force tending to oppose rotational movement of the hand about the carpus.”

Finger Restraint

This device restrains the fingers;

U.S. Pat. No. 5,965,783 (Klimoski): This is “a finger restraint device . . . which will support the wearer's fingers in a curled position”.

Other prior art includes D521,644 S (Nordt et al.): This is a design patent for an ornament on a support brace.

What is needed is a device that reminds the wearer to place his or her hands in a position to prevent carpal tunnel syndrome, that does not itself restrict flexion or finger movement. The device needs to discourage but not prevent extension, and easily breaks away to allow full extension.

### SUMMARY OF THE INVENTION

In accordance with the invention, a device worn on the wrist discourages extension but enable flexion of the wrist. The device is adapted to break away or release under sufficient extension force, so that the user is not prevented from extending the wrist should it be necessary or desired.

Accordingly, it is an object of the present invention to provide an improved device for encouraging proper wrist position.

It is a further object of the present invention to provide an improved device that encourages proper wrist position but does not prevent necessary wrist movement.

It is yet another object of the present invention to provide an improved device to remind a user not to overly extend the wrist.

The subject matter of the present invention is particularly pointed out and distinctly claimed in the concluding portion of this specification. However, both the organization and method of operation, together with further advantages and objects thereof, may best be understood by reference to the following description taken in connection with accompanying drawings wherein like reference characters refer to like elements.

### BRIEF DESCRIPTION OF THE DRAWINGS

FIG. 1 is a side view of the device in accordance with the invention as worn on a user's wrist;

FIG. 2 is a side view showing how the breakaway feature functions as the wrist bends.

FIG. 3 is a side view of a wrist band;

FIG. 4 is an end view of the wrist band of FIG. 3 looking in the direction of arrow 4;

FIG. 5 is a bottom view of a bar portion;

FIG. 6 is a view of an alternate bar portion configuration;

FIG. 7 is a view of another alternate bar portion configuration;

FIG. 8 is a perspective view of an alternate configuration; and

FIG. 9 is an end view of the configuration of FIG. 8.

### DETAILED DESCRIPTION

The system according to a preferred embodiment of the present invention comprises a device worn on the wrist that is to remind the user to maintain the wrist in an appropriate position to minimize aggravation of carpal tunnel syndrome, for example.

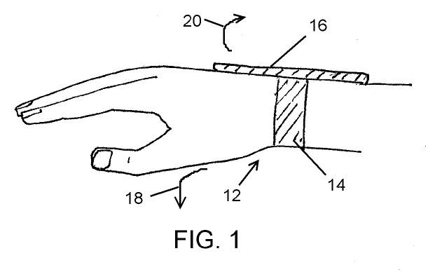

Referring to FIG. 1, a side view of the device as worn on the wrist 12 of a user, the device includes 2 parts, a part 14 that holds onto the wrist (called a wristband here) and an elongate bar portion 16 that is rigid or semi-rigid that is positioned on the top side of the wrist and extends in the direction of the axis of length of the arm. The bar portion, as held in position by the wristband, thus allows downward flexing of the wrist in the direction of arrow 18, but, will provide resistance to upward extension of this wrist in the direction of arrow 20. This will help to remind the user to keep the wrist in a position that is less likely to cause or aggravate carpal tunnel syndrome or repetitive stress injury.

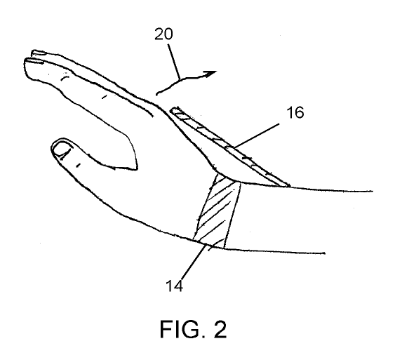

The device prevents upward flexing of the wrist under slight pressure by interaction of the upper surface of the hand with the bar 16, but for safety purposes, is designed to release under greater pressure. The release may be accomplished either by the bar detaching from the wristband, as illustrated in FIG. 2, a side view of the device showing bar 16 coming loose from the band 14 as the hand is moved upwardly in the direction of arrow 20 relative to the wrist. Alternatively, the wristband 14 may be designed so as to let go of the wrist.

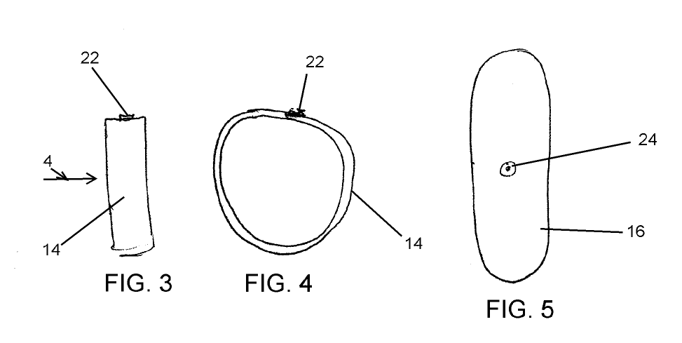

Referring now to FIGS. 3-5, which are a side view and end view of a wrist band and a bottom view of a bar portion, respectively, the wrist band 14 suitably includes a releasable fastener portion 22 mounted thereon in a position suitably positionable at the top of the user's wrist when wearing the band 14, and bar 16 includes a corresponding fastener portion 24 on the bottom face thereof. In use, in the configuration shown in FIG. 1, portions 22 and 24 engage so as to secure the bar 16 to the wrist band 14. The fasteners 22, 24 together comprise breakaway fastening devices, and may be, for example, corresponding portions of snap fasteners or, hook-and-loop type fasteners such as Velcro brand fasteners, enabling connection together to hold the bar and wrist band together, but, releasing the connection under a certain amount of force.

The releasing function provides a safety or convenience feature, whereby should the user need to extend the wrist for whatever reason, the fasteners 22, 24 will release and the bar and band will separate (as in FIG. 2) so the user's movement of the wrist is not impeded.

The device can be made in a number of ways. The wristband can either completely or partially encircle the wrist and can be made of fabric, plastic or metal, for example, and may be elastic.

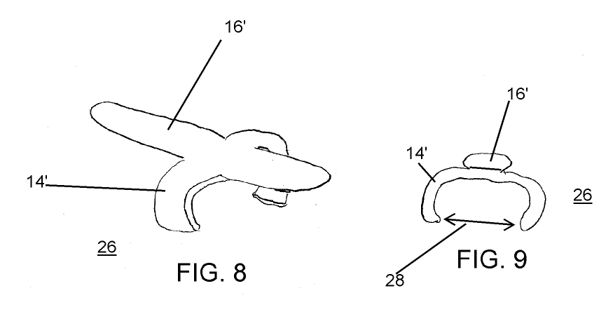

In an alternate configuration, referring to FIG. 8 and FIG. 9, which are perspective and end views of a device 26 wherein wrist band portion 14′ and bar 16′ are integrally formed and band portion 14′ partially surrounds the wrist of the user such that under force of extension of the wrist, the band portion 14′ (which may be flexible to a certain degree to enable the distance 28 between the opposite legs of the band portion to expand by outward flexing of the legs) will let go of or otherwise disengage from the wrist to allow the entire device to break free from the user. In the configuration of FIGS. 8 and 9, bar 16′ is integrally formed as a portion of the band 14′, but can also be designed to be removable or detachable from the band 14′.

The bar 16, 16′ can be made of any material, and a typical length of the bar is 4 inches in a preferred embodiment. The width of the bar is less of a factor, but may be 1 inch, for example. The bar can be made in a decorative form, e.g. the shape of a flower, butterfly or other object to provide an aesthetically pleasing appearance. It can be substantially flat, or made with somewhat 3D configuration to provide some additional aesthetically pleasing appearance.

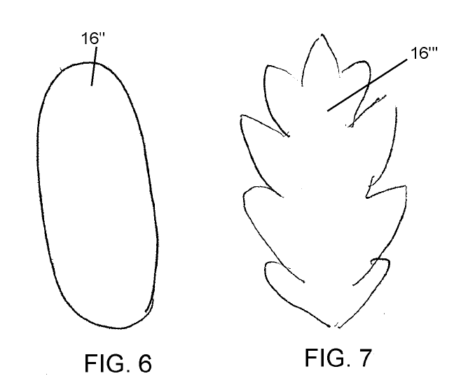

FIG. 6 and FIG. 7, top views of alternative embodiments of the breakaway device, show a somewhat more oval shape bar 16″ (FIG. 6) or a leaf-like appearance bar 16′″ (FIG. 7). Other decorative shapes, such as a butterfly or flower, etc., may be employed, so long as the shape fits relative to the user's wrist.

In accordance with the invention, an improved device is provided for training the wearer in and reminding the wearer of proper wrist position when typing or the like. When compared with the prior art devices that immobilize the wrist, the present invention provides an improvement in that the prior devices do not break away and restrict wrist movement. The present invention allows full wrist flexion. It discourages wrist extension but does not prevent it, and breaks away with slight pressure. Unlike the finger restraint devices, the fingers are not prevented from free movement by the present invention.

Thus, the present device prevents upward extension of the wrist under slight pressure, but for safety purposes, is designed to release under greater pressure. The release may be accomplished either by the bar detaching from the wristband, or the wristband letting go of the wrist. The device can be made to either completely or partially encircle the wrist, made of fabric, plastic or metal, for example, and may be elastic. The bar can be made of any material and shape so long as it fits the size parameters and is at least semi-rigid, and can attach firmly to the wristband, or can be designed to easily detach by using a weak bonding (hook-and-loop, snap connection, etc.).

The bar may be generally 3″ to 4″ long, and ½″ to 1½″ wide. The attachment is fastened in a “breakaway” mode, so that mild pressure will cause it to detach. This is for safety reasons since, for example, if the wearer is in motion and stumbles or falls, the device will break away and allow full extension of the wrist.

If the wearer flexes the wrist upward, the rigid device acts as a reminder to keep the wrist in a level position.

This invention is of particular use to persons using a computer keyboard, those playing musical instruments such as the piano or violin, and those engaging in occupations or hobbies in which it is common to bend the hand upward while moving the fingers.

While a preferred embodiment of the present invention has been shown and described, it will be apparent to those skilled in the art that many changes and modifications may be made without departing from the invention in its broader aspects. The appended claims are therefore intended to cover all such changes and modifications as fall within the true spirit and scope of the invention.

What is claimed is:

1. A wrist position training device, comprising:
an arm engaging portion for attachment to a wearer's arm; and
a wrist movement inhibitor cooperating with said arm engaging portion for discouraging wrist extension beyond a position,
said wrist position training device allowing wrist extension beyond said position when said extension is made with a force beyond a value.

2. The wrist position training device according to claim 1, wherein said wrist movement inhibitor comprises a member removably attached to said arm engaging portion, said member having an elongate axis aligned substantially with a length axis of the wearer's arm.

3. The wrist position training device according to claim 1, wherein said wrist movement inhibitor comprises a member in breakaway engagement with said arm engaging portion, said member breaking away in presence of extension force beyond said value.

4. The wrist position training device according to claim 3, wherein said member is removably attached to said arm engaging portion by use of a snap.

5. The wrist position training device according to claim 3, wherein said member is removably attached to said arm engaging portion by use of hook and loop fasteners.

6. The wrist position training device according to claim 1, wherein said arm engaging portion comprises an elastic band adapted for wearing around the wearer's wrist.

7. The wrist position training device according to claim 1, wherein said arm engaging portion comprises a C-shaped wrist engaging member for attachment about the wearer's wrist.

8. The wrist position training device according to claim 7, wherein portions of said wrist engaging member are flexible to enable detachment from the wearer's wrist in presence of the force beyond a value.

9. The wrist position training device according to claim 7, wherein said wrist movement inhibitor comprises a member attached to said arm engaging portion, said member having a portion aligned substantially with a length axis of the wearer's arm.

10. A wrist safety device, comprising:
a band for attachment to a wearer's wrist; and
a movement inhibitor attached to said band, said movement inhibitor having a portion extending at least from the wearer's wrist along a portion of an upper part of the wearer's hand for discouraging extension of the wearer's wrist,
wherein said movement inhibitor is detachably engaged to said band position and will detach therefrom in presence of an extension force beyond a value.

11. The wrist safety device according to claim 10, wherein said movement inhibitor comprises an elongate bar member.

12. The wrist safety device according to claim 10, further comprising a snap having first and second engaging portions, one of said first and second portions being secured to said band and the other of said first and second portions being engaged to said elongate bar member.

13. The wrist safety device according to claim 10, further comprising first and second hook and loop fastener portions, one of said first and second portions being secured to said band and the other of said first and second portions being engaged to said elongate bar member.

14. The wrist safety device according to claim 10, wherein said elongate bar member has an elongate portion thereof aligned substantially with a length axis of the wearer's arm.

15. A method of teaching appropriate wrist positioning, comprising:
providing a wrist extension inhibitor to discourage a wearer's wrist extension beyond a position; and
withdrawing the wrist extension inhibitor in presence of an extension force beyond a value.

16. The method of teaching appropriate wrist positioning according to claim 15, wherein said wrist extension inhibitor is removably engaged to the wearer's wrist and detaches therefrom in presence of said extension force beyond a value.

17. The method of teaching appropriate wrist positioning according to claim 15, wherein said providing step comprises attaching an elongate member to the wearer's wrist.

18. The method of teaching appropriate wrist positioning according to claim 18, wherein said withdrawing step comprises detaching said elongate member from the wearer's wrist.
